(19) United States
(12) Patent Application Publication
(10) Pub. No.: US 2010/0280261 A1
(43) Pub. Date: Nov. 4, 2010

(54) POROUS BODY PRECURSORS, SHAPED POROUS BODIES, PROCESSES FOR MAKING THEM, AND END-USE PRODUCTS BASED UPON THE SAME

(75) Inventors: Kevin E. Howard, Midland, MI (US); Cathy L. Tway, Midland, MI (US); Peter C. Lebaron, Hope, MI (US); Jamie L. Lovelace, Bay City, MI (US); Hirokazu Shibata, Bergen op Zoom (NL)

Correspondence Address: THE DOW CHEMICAL COMPANY/KSJLAW, P.O. BOX 59, MARINE ON ST. CROIX, MN, 55047, US

(73) Assignee: DOW TECHNOLOGY INVESTMENTS LLC, Midland, MI (US)

(21) Appl. No.: 12/757,984

(22) Filed: Apr. 10, 2010

Related U.S. Application Data
(60) Provisional application No. 61/173,643, filed on Apr. 29, 2009.

(51) Int. Cl. C07D 317/36; B01J 23/14; B01J 31/06; B01J 23/68; B01J 23/36; C07D 301/10; C07C 29/00; C07C 213/02; C07C 41/03

(52) U.S. Cl. 549/230; 502/349; 502/159; 502/347; 549/536; 568/867; 568/704; 568/672

(57) ABSTRACT

The present invention provides porous body precursors and shaped porous bodies. Also included are catalysts and other end-use products based upon the shaped porous bodies and thus the porous body precursors. Finally, processes for making these are provided. The porous body precursors are germanium doped and comprise a precursor alumina blend. It has now surprisingly been discovered that inclusion of germanium, alone or in combination with such a blend, in porous body precursors can provide control over, or improvements to, surface morphology, physical properties, and/or surface chemistry of shaped porous bodies based thereupon. Surprisingly and advantageously, heat treating the shaped porous bodies can result in additional morphological changes so that additional fine tuning of the shaped porous bodies is possible in subsequent steps.

### FIELD OF THE INVENTION

The present invention provides porous body precursors and shaped porous bodies. Also included are catalysts and other end-use products, such as filters, membrane systems, composite bodies, insulating materials, and the like, based upon the shaped porous bodies and thus the porous body precursors. Finally, processes for making these, and further downstream products, are also provided.

### BACKGROUND

Many facets of the practice of chemistry and/or chemical engineering can be reliant upon providing structures or surfaces capable of performing or facilitating separations or reactions and/or providing areas for such separations or reactions to take place. Such structures or surfaces are thus ubiquitous in many R&D and manufacturing settings. Although the desired physical and chemical properties of these shaped bodies can, and will, vary depending on the particular application, there are certain properties that are generally desirable in such shaped bodies regardless of the final application in which they will be utilized.

For example, such shaped bodies will desirably be of high purity and substantially inert so that the shaped bodies themselves will not participate in the separations or reactions taking place around, on or through them in a way that is undesired, unintended, or detrimental. For those shaped bodies for which it is desired to have the components being reacted or separated pass through, or diffuse into, the shaped body, a low diffusion resistance would be advantageous. In certain applications, the shaped bodies are desirably provided within a reaction or separation space, and so they are desirably of sufficient mechanical integrity to avoid being crushed, chipped or cracked during transport or placement. For those shaped bodies desirably utilized as reaction surfaces, high surface area and/or high porosity can be desired, to improve the loading and dispersion of the desired reactants, and also to provide enhanced surface area on which the reactions or separations can take place. Of course, in almost every application, lower cost materials are preferred.

Oftentimes, the desired properties of such shaped bodies can conflict with one another, and as a result, preparing shaped bodies where each desired property is maximized can be challenging. In efforts to meet these challenges, much research has been conducted not only on the components and additives utilized in the bodies, but also on the physical properties of shaped bodies so formed. However, many of the shaped porous bodies developed to date have yet to provide the full spectrum of desired properties for these materials. And, once conventional shaped porous bodies, their properties may not typically be altered, but for via the impregnation thereupon of additional modifiers.

Desirably, porous body precursors and shaped porous bodies would be provided that could optimize a plurality of properties, or at least optimize at least one property without substantial detriment to another. Such shaped porous bodies would provide improvements to products, e.g., catalysts, in which they were used.

### BRIEF DESCRIPTION

The present invention provides such improvements to porous body precursors and shaped porous bodies as well as the processes for producing them. Specifically, the present invention provides germanium-doped porous body precursors, upon which shaped porous bodies may be based. In some embodiments, the porous body precursors comprise a precursor alumina blend. It has now surprisingly been discovered that inclusion of germanium, alone or in combination with such a blend, in porous body precursors can provide control over, or improvements to, surface morphology, physical properties, and/or surface chemistry of shaped porous bodies based thereupon. Surprisingly and advantageously, heat treating the shaped porous bodies can result in additional morphological changes so that additional fine tuning of the shaped porous bodies is possible in subsequent steps.

In a first aspect, the present invention provides a germanium-doped porous body precursor comprising a precursor alumina blend. The precursor alumina blend may comprise at least two secondary particle sizes of one precursor alumina, or, may comprise at least two precursor aluminas being of substantially of the same secondary particles size, or may comprise at least two precursor aluminas of differing secondary particle sizes. The precursor aluminas may comprise any transition alumina precursor, transition alumina, alpha-alumina precursors, and as such, may comprise gibbsite, bayerite, and nordstrandite, boehmite, pseudo-boehmite, diaspore, gamma-alumina, delta-alumina, eta-alumina, kappa-alumina, chi-alumina, rho-alumina, theta-alumina, aluminum trihydroxides and aluminum oxide hydroxides. The porous body precursors desirably comprise transition alumina precursors, transition aluminas, alpha-alumina precursors, or combinations of these.

Because the precursor alumina blend and/or germanium may be so effective at providing properties to, or enhancing properties of, shaped porous bodies prepared from porous body precursors comprising the blend and/or germanium, the use of additional modifiers or additives can be reduced or substantially avoided. Rather, and surprisingly, any desired fine tuning of the shaped porous bodies may be provided by subjecting the shaped porous bodies to a heat treatment after their processing is otherwise complete, an ability not provided by other dopants or additives. A second aspect of the invention thus provides a shaped porous body prepared from a germanium-doped porous body precursor comprising a precursor alumina blend. The shaped porous body may desirably comprise alpha alumina, and in some embodiments, fluoride affected alpha-alumina. In such embodiments, the shaped porous body may have been subjected to a heat treatment, so that the resulting heat treated shaped porous body has an increased crush strength, porosity, beneficial morphological change, and/or improved pore size distribution relative to the shaped porous body prior to the heat treatment, or even shaped porous bodies subjected to such a heat treatment but based upon porous body precursors not comprising germanium.

In a third aspect, methods of providing the shaped porous bodies are also provided, and comprise preparing a germanium-doped porous body precursor, processing the porous body precursor into the shaped porous body, and exposing the shaped porous body to a heated oxidative atmosphere. The atmosphere may be at least about 1000° C., at least about 1200° C., or even at least about 1400° C. The processing may include exposing the porous body precursor and/or the shaped porous body to a fluorine-containing species.

The improved morphological and/or physical properties that can be provided to the shaped porous bodies by virtue of the inclusion of the germanium and/or the precursor alumina blend in the porous body precursors, the shaped porous bodies are expected to be advantageously employed in many end-use applications. In a fourth aspect, the present invention contemplates such use, and provides rhenium-promoted catalysts based upon the shaped porous bodies. More specifically, the rhenium-promoted catalysts comprise at least one catalytic species deposited on the shaped porous bodies, wherein the shaped porous bodies are prepared from germanium-doped porous body precursors. In some embodiments, the porous body precursors may also comprise a precursor alumina blend. Exposing the shaped porous bodies to a heated oxidative atmosphere may provide additional, or enhanced, morphological or physical properties to the shaped porous bodies and thus the catalysts, and is contemplated in some embodiments. The catalytic species may preferably comprise a silver component.

A process for making a rhenium-promoted catalyst is provided in a further aspect, the process comprising selecting a shaped porous body prepared from a germanium-doped porous body precursor, and in some embodiments, a precursor alumina blend. At least one catalytic species is then deposited on the shaped porous bodies to provide catalysts. The process may further comprise exposing the shaped porous bodies, or catalysts, as the case may be, to a heated oxidative or non-oxidative atmosphere prior to, or after, deposition of the catalytic species.

The advantageous properties provided by the germanium-doped porous body precursors to the shaped porous bodies are expected to translate into improvements in one or more catalyst properties, which in turn, are expected to provide improvements to the processes in which the catalysts are utilized. As a result, and in yet another aspect, the present invention provides a process for the epoxidation of an alkylene. The process comprises reacting a feed comprising one or more alkylenes and oxygen in the presence of the catalyst based upon a shaped porous body which in turn is based upon a germanium-doped porous body precursor, and in some embodiments, a precursor alumina blend.

The advantages provided to such processes can be further leveraged by utilization of the alkylene oxides produced thereby in further downstream processes, and such processes are thus provided in yet another aspect of the invention. More specifically, the present invention also provides a process for preparing a 1,2-diol, a 1,2-diol ether, a 1,2-carbonate, or an alkanolamine. The process comprises converting an alkylene oxide into the 1,2-diol, 1,2-diol ether, a 1,2-carbonate, or alkanolamine, wherein the alkylene oxide is prepared by a process utilizing a catalyst based upon a shaped porous body, which in turn, is based upon a germanium-doped porous body precursor, and in some embodiments, a precursor alumina blend.

### DRAWINGS

These and other features, aspects, and advantages of the present invention will become better understood when the following detailed description is read with reference to the accompanying drawings in which like characters represent like parts throughout the drawings, wherein:

FIG. 1A is a scanning electron micrograph of a random sample of a comparative shaped porous body, not comprising germanium dopant, or a precursor alumina blend;

FIG. 1B is a scanning electron micrograph of a random sample of a comparative shaped porous body, not comprising dopant, or a precursor alumina blend after heat treatment at 1400° C. for two hours;

FIG. 2A is a scanning electron micrograph of a random sample of an inventive shaped porous body, based upon a germanium doped porous body precursor (Example 1, Sample #1);

FIG. 2B is a scanning electron micrograph of a random sample of an inventive shaped porous body, based upon a germanium doped porous body precursor (Example 1, Sample #1), after heat treatment at 1400° C. for two hours;

FIG. 3A is a scanning electron micrograph of a random sample of an inventive shaped porous body, based upon a germanium doped porous body precursor (Example 1, Sample #2);

FIG. 3B is a scanning electron micrograph of a random sample of an inventive shaped porous body, based upon a germanium doped porous body precursor (Example 1, Sample #2), after heat treatment at 1400° C. for two hours;

FIG. 4A is a scanning electron micrograph of a random sample of an inventive shaped porous body, based upon a germanium doped porous body precursor (Example 1, Sample #3); and

FIG. 4B is a scanning electron micrograph of a random sample of an inventive shaped porous body, based upon a germanium doped porous body precursor (Example 1, Sample #3), after heat treatment at 1400° C. for two hours.

### DETAILED DESCRIPTION

The present specification provides certain definitions and methods to better define the present invention and to guide those of ordinary skill in the art in the practice of the present invention. Provision, or lack of the provision, of a definition for a particular term or phrase is not meant to imply any particular importance, or lack thereof. Rather, unless otherwise defined, technical and scientific terms used herein have the same meaning as is commonly understood by one of skill in the art to which this invention belongs.

The terms “first”, “second”, and the like, as used herein do not denote any order, quantity, or importance, but rather are used to distinguish one element from another. Also, the terms “a” and “an” do not denote a limitation of quantity, but rather denote the presence of at least one of the referenced item, and the terms “front”, “back”, “bottom”, and/or “top”, unless otherwise noted, are merely used for convenience of description, and are not limited to any one position or spatial orientation. If ranges are disclosed, the endpoints of all ranges directed to the same component or property are inclusive and independently combinable (e.g., a range of “up to about 25 wt. %, or, more specifically, about 5 wt. % to about 20 wt. %,” is inclusive of the endpoints and all intermediate values of the ranges of “about 5 wt. % to about 25 wt. %,” etc.). The modifier “about” used in connection with a quantity is inclusive of the stated value and has the meaning dictated by the context (e.g., includes at least the degree of error associated with measurement of the particular quantity). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described inventive features may be combined in any suitable manner in the various embodiments.

The present invention provides germanium-doped porous body precursors, comprising a blend of one or more precursor aluminas. Advantageously, the germanium and/or precursor alumina blend may provide morphological, other physical property, and/or surface chemistry enhancements to the porous body precursors, or shaped porous bodies or catalysts etc., based upon the same. In some embodiments, the shaped porous bodies may be subjected to a post-processing heat treatment that can provide further such enhancements, providing the opportunity to further fine tune the properties.

As used herein, the phrase “porous body precursor” is defined as a solid which has been formed into a selected shape suitable for its intended use, and in which shape it will be calcined or otherwise processed or reacted to provide a shaped porous body. The phrase, “shaped porous body”, in turn, is meant to indicate a solid which has been formed into a selected shape suitable for its intended use and has been further processed so as to have a porosity of greater than at least about 10%. As those of ordinary skill in the art are aware, shaped porous bodies may typically be comprised of many, typically thousands, tens of thousands, hundreds of thousands or even millions of smaller particles, and typically, in the present application, it is the surface morphology or aspect ratio of these smaller particles that is observed or measured and referred to herein. As such, it is to be understood that when particular ranges are indicated as advantageous or desired for these measurements, or that a particular surface morphology has been observed, that these ranges may be based upon the measurement or observation of from about 1 to about 10 particles, and although it may generally be assumed that the majority of the particles may thus exhibit the observed morphology or be within the range of aspect ratio provided, that the ranges are not meant to, and do not, imply that 100% of the population, or 90%, or 80%, or 70%, or even 50% of the particles need to exhibit a surface morphology or possess an aspect ratio within this range.

As used herein, the phrase “precursor aluminas” is meant to include transition alumina precursors, transition aluminas, and other alpha-alumina precursors. “Transition alumina precursors”, in turn, are one or more materials that, upon thermal treatment, are capable of being at least partially converted to alpha alumina. Transition alumina precursors include, but are not limited to, aluminum tri-hydroxides, such as gibbsite, bayerite, and nordstrandite, aluminum oxide hydroxides, such as boehmite, pseudo-boehmite and diaspore. “Transition aluminas” are one or more aluminas other than alpha-alumina, which are capable of being at least partially converted to alpha-alumina under thermal treatment at 900° C. or greater. Transition aluminas possess varying degrees of crystallinity, and include, but are not limited to, gamma-alumina, delta-alumina, eta-alumina, kappa-alumina, chi-alumina, rho-alumina, and theta-alumina. “Alpha-alumina precursor” means one or more materials capable of being transformed into alpha-alumina, including transition alumina precursors and transition aluminas. Further, as used herein, the phrase “secondary particle” means an aggregate of primary particles of a precursor alumina. Primary particles of precursor aluminas are individual crystallites of the precursor aluminas and are typically on the order of nanometers in size and as such, are typically most accurately measured by x-ray diffraction. Secondary particles are aggregates of at least two of these primary particles, have sizes on the order of micrometers, and may be most accurately measured by light-scattering or sedimentation methods.

The germanium and/or selected blend of precursor aluminas may enhance at least one property of a shaped porous body prepared from a porous body precursor comprising the germanium and/or blend. Any property desirably enhanced in such shaped porous bodies is within the scope of the present invention, and such properties may typically include surface area, particle aspect ratio, pore volume, median pore diameter, surface morphology, crush strength, yield or failure stress, calcined density, etc. “Surface area”, as used herein, refers to the surface area of the shaped porous bodies as determined by the BET (Brunauer, Emmett and Teller) method by nitrogen as described in the Journal of the American Chemical Society 60 (1938) pp. 309-316. “Aspect ratio” means the ratio of the longest or major dimension to the smallest or minor dimension of the particles of which the shaped porous bodies are comprised, determined by examination of the scanning electron micrograph of the shaped porous body. “Pore volume” (also, “total intrusion volume”) means pore volume of the shaped porous body and is typically determined by mercury porosimetry. The measurements reported herein used the method described in Webb & Orr, Analytical Methods in Fine Particle Technology (1997), p. 155, using mercury intrusion to 60,000 psia using Micrometrics Autopore IV 9520, assuming 130° contact angle, 0.473 N/M surface tension of Hg. “Median pore diameter” means the pore diameter corresponding to the point in the pore size distribution at which half of the cumulative pore volume of the shaped porous body has been measured, and ‘surface morphology’ means the physical structure of the surface of the particles of which the shaped porous body is comprised, typically observed by scanning electron microscopy (SEM). Crush strength can be determined according to ASTM Method No. D-6175-98. Yield or failure stress can be determined according to ASTM C 1161-94.

A germanium-doped porous body precursor, as used herein, indicates a porous body precursor comprising an amount of germanium. While not wishing to be bound by any theory, it has been discovered that germanium, alone or in some embodiments, in combination with a precursor alumina blend, can provide, or enhance, physical, morphological or surface chemistry properties of a shaped porous body based upon a porous body precursor comprising the germanium and a form of alumina. Further, these properties may be further adjusted, or “fine tuned”, via a heat treatment after the precursor has been processed to form the shaped porous body, an ability not provided by any other modifier or dopant known to the Applicants.

The germanium (Ge) may be provided in any form. For example, the germanium may be provided as a solid such as germanium oxide (GeO₂), germanium chloride (GeCl), germanium oxychloride, other germanium halides such as germanium bromide or germanium iodide. Alternatively, the germanium may be provided in a gaseous phase introduction, using for example GeH₄, Ge₂H₆, or GeF₄. Such a gas phase introduction may be concomitant with, or subsequent to, a fluoride affectation step, if utilized.

Any amount of the germanium-containing composition may be included in the inventive porous body precursors. Practicality may dictate that only as much of the germanium-containing composition should be used to achieve the maximum effect, and not so much as to unnecessarily add to the cost, or detrimentally impact the processability of the porous body precursors. That being said, the germanium-containing composition of the present invention advantageously can exert its effects in surprisingly low amounts, and it is expected that amounts of less than 10 weight percent (wt %) based upon the total weight of the porous body precursor, or less than 5 wt %, or less than 3 wt %, or even less than 1%, will be required to provide appreciable enhancements.

Any combination of precursor aluminas, or particles sizes of one or more precursor aluminas, capable of providing a desired property to, or enhancing a property of, porous body precursors and/or shaped porous bodies is considered to be within the scope of the present invention. In particularly advantageous embodiments of the invention, the precursor aluminas selected for use in the blend may act synergistically to provide properties, or enhancements to properties, in the shaped porous bodies that are greater than the weighted average of the properties in shaped porous bodies prepared from either precursor alumina alone.

As mentioned above, the blend of precursor aluminas may comprise blends of one (in those embodiments of the invention wherein the blend comprises a blend of multiple secondary particle sizes of a single precursor alumina) or more transition alumina precursors, transition aluminas, or alpha-alumina precursors. The blend of precursor aluminas may thus comprise a blend of one or more gibbsites, bayerites, nordstrandites, boehmites, pseudo-boehmites, diaspores, gamma-aluminas, delta-aluminas, eta-aluminas, kappa-aluminas, chi-aluminas, rho-aluminas, theta-aluminas, aluminum trihydroxides and aluminum oxide hydroxides. Preferred blends comprise blends of one or more gibbsites and/or pseudo-boehmites.

As those of ordinary skill in the art are aware, the aforementioned transition alumina precursors, transition aluminas and alpha-alumina precursors may include numerous variants. Furthermore, these variants, conventionally differentiated by tradenames (e.g., Catapal B vs Catapal D, Versal V-250 vs Versal V-700) may differ only incrementally in chemical composition, physical and/or mechanical properties, such as density, pore volume, surface area, secondary particle size and primary, or crystallite, particle size. Yet, it has now been surprisingly discovered that precursor alumina blends comprising two or more variants of one type of transition alumina precursor, transition alumina, or alpha-alumina, or even two secondary particle sizes of a single variant, may yet provide a porous body precursor with properties synergistically enhanced relative to those comprising either variant, or either particle size of the variant, alone. As such, precursor alumina blends comprising two, e.g., pseudo-boehmite, gibbsite, boehmite, variants and porous body precursors, shaped porous bodies and end-use products based upon the same are considered to be within the scope of the invention. The nomenclature and properties of precursor aluminas are discussed at length in “Oxides and Hydroxides of Aluminum”, Alcoa Technical Paper No. 19, Wefers and Misra, Alcoa Laboratories, 1987, commercially available for download at http://www.alcoa.com/global/en/innovation/papers_patents/details/1987_paper_oxides_a nd_hydroxides.asp# and incorporated by reference herein for any and all purposes.

The precursor alumina blend may comprise any ratio of the selected precursor aluminas (or secondary particle sizes of a single precursor alumina) that provides an improvement to a property of shaped porous bodies prepared from porous body precursors comprising the blend. The selected precursor aluminas may be provided in substantially equal amounts, or, a majority of one may be provided. Exemplary ratios for blends comprising two precursor aluminas, or two secondary particle sizes of one precursor alumina, may thus range from 1:1, to as much as 100:1. Typically, ranges of from 1:1 to 10:1, or from 1:1 to 5:1 may be employed. In those preferred embodiments of the invention wherein the precursor alumina blend comprises more than two precursor aluminas, the ratio of aluminas may be such that the aluminas are present in relatively equal amounts, one or more are in a majority, one or more are in the minority, etc. Thus, suitable ratios for these blends may be from about 1:1:1 (or 1:1:1:1, etc.) to about 100:1:1 (or 100:1:1:1, etc) or from about 1:1:1 to about 10:1:1 (or 10:1:1:1, etc.), or from about 1:1:1 to about 5:1:1 (or 5:1:1:1, etc)

In addition to the germanium and/or precursor alumina blend, the porous body precursors may comprise additional porous refractory structure or support materials, so long as whatever the additional porous refractory material(s) chosen, it is relatively inert in the presence of the chemicals and processing conditions employed in the application in which the shaped porous body will be utilized. In addition to the precursor alumina blend, the porous body precursors may comprise, if desired, silicon carbide, silicon dioxide, titania, zirconia, zirconium silicate, graphite, magnesia and various clays. If the porous body precursors desirably comprise other support materials, they are desirably present in relatively minor amounts, i.e., the precursor alumina blend may make up at least 50 wt %, or even 65 wt %, or up to about 75 wt %, of the porous body precursors. In preferred embodiments, the porous body precursors are comprised entirely of the precursor alumina blend.

The porous body precursors of the invention may comprise any other components, in any amounts, necessary or desired for processing, such as, e.g., water, acid, binders, pore formers, dopants, etc., of common knowledge to those of ordinary skill in the art of the production of shaped porous bodies for use as structures or supports. In those embodiments of the invention wherein the porous body precursors are intended for use in shaped porous bodies that will ultimately be used in catalytic applications, the porous body precursors may also contain precursor catalyst compounds that have elements that may desirably be incorporated onto the surface, at crystalline grain boundaries or into the lattice structure of the alpha-alumina particles that will be formed upon processing of the porous body precursors to form shaped porous bodies. Examples of compounds useful for forming these incorporated catalysts include inorganic and organic compounds that form catalysts such as metals, metal oxides, metal carbides, metal nitrides and organo-metallic compounds.

The porous body precursors may also comprise other organic compounds (e.g., binders and dispersants, such as those described in Introduction to the Principles of Ceramic Processing, J. Reed, Wiley Interscience, 1988) to facilitate the shaping, or to alter the porosity, of the porous body precursors and/or shaped porous bodies. Pore formers (also known as burn out agents) are materials used to form specially sized pores in the shaped porous bodies by being burned out, sublimed, or volatilized. Pore formers are generally organic, such as ground walnut shells, paraffin wax, petroleum jelly, granulated polyolefins, such as polyethylene and polypropylene, but examples of inorganic pore formers are known. The pore formers are usually added to the porous body precursor raw materials prior to shaping. During a drying or calcining step or during the conversion of the alpha-alumina precursor to alpha-alumina, the pore formers are burned out, sublimed, or volatilized.

The germanium and/or precursor alumina blends identified herein may prove so effective at imparting the desired properties, or enhancements to the property(ies), that the use of additional modifiers for this purpose may be reduced or substantially avoided. Nonetheless, if the same is desired or required, modifiers may also be added to the porous body precursor raw materials or the porous body precursors to change the chemical and/or physical properties of the shaped porous bodies or end-use products based upon the shaped porous bodies. If inclusion of the same is desired or required, any chosen modifier(s) can be added during any stage of the process, or at one or more steps in the process. For example, a metal oxide modifier can be added to the porous body precursor raw materials prior to, or after, the mixing/mulling step, prior to, or after, formation of the porous body precursors into formed porous body precursors, or before or after drying, or other thermal processing of the shaped porous bodies.

As used herein, “modifier” means a component other than the precursor alumina blend, and any other optional porous refractory material, added to a porous body precursor or shaped porous body to introduce desirable properties such as improved end-use performance. More particularly, modifiers can be inorganic compounds or naturally occurring minerals which are added in order to impart properties such as strength and, in some cases, change the surface chemical properties of the shaped porous bodies and/or end use products based thereupon. Non-limiting examples of such modifiers include zirconium silicate, see WO 2005/039757, alkali metal silicates and alkaline earth metal silicates, see WO 2005/023418, each of these being incorporated herein by reference for any and all purposes, as well as metal oxides, mixed metal oxides, for example, oxides of cerium, manganese, tin, and rhenium.

Whatever the raw materials selected for use in the porous body precursors, they are desirably of sufficient purity so that there are limited reactions between any of them. In particular, the precursor alumina blend should be of sufficient purity so that any impurities are not present in a quantity sufficient to substantially detrimentally impact the properties of the porous body precursors, shaped porous bodies and/or catalysts, i.e., any impurities are desirably limited to not more than 3 wt %, or even not more than 1.5 wt %, of the total weight of the porous body precursors.

The desired components of the porous body precursors, i.e., at least the germanium and/or precursor alumina blend, may be combined by any suitable method known in the art. Further, the precursor alumina blend and any other desired raw materials may be in any form, and combined in any order, i.e., the order of addition of the precursor alumina blend to the other raw materials, and the order of addition of the precursor aluminas themselves to the blend, is not critical. Examples of suitable techniques for combining the porous body precursor materials include ball milling, mix-mulling, ribbon blending, vertical screw mixing, V-blending, and attrition milling. The mixture may be prepared dry (i.e., in the absence of a liquid medium) or wet.

Once mixed, the porous body precursor materials may be formed by any suitable method, such as e.g., injection molding, extrusion, isostatic pressing, slip casting, roll compaction and tape casting. Each of these is described in more detail in Introduction to the Principles of Ceramic Processing, J. Reed, Chapters 20 and 21, Wiley Interscience, 1988, incorporated herein by reference in its entirety for any and all purposes. Suitable shapes for porous body precursors will vary depending upon the end use of the same, but generally can include without limitation pills, chunks, tablets, pieces, spheres, pellets, tubes, wagon wheels, toroids having star shaped inner and outer surfaces, cylinders, hollow cylinders, amphora, rings, Raschig rings, honeycombs, monoliths, saddles, cross-partitioned hollow cylinders (e.g., having at least one partition extending between walls), cylinders having gas channels from side wall to side wall, cylinders having two or more gas channels, and ribbed or finned structures. If cylinders, the porous body precursors may be circular, oval, hexagonal, quadrilateral, or trilateral in cross-section. In those embodiments of the invention wherein the porous body precursors are used to prepare shaped porous bodies intended for end use as catalysts, the porous body precursors may desirably be formed into a rounded shape, e.g., pellets, rings, tablets and the like, having diameters of from about 0.1 inch (0.25 cm) to about 0.8 inch (2 cm).

The porous body precursors so formed may then optionally be heated under an atmosphere sufficient to remove water, decompose any organic additives, or otherwise modify the porous body precursors prior to introduction into a kiln, oven, pressure-controlled reaction vessel or other container for any further required processing into shaped porous bodies. Suitable atmospheres include, but are not limited to, air, nitrogen, argon, hydrogen, carbon dioxide, water vapor, those comprising fluorine-containing gases or combinations thereof.

Before or during calcination, and in those embodiments of the invention wherein the porous body precursors comprise one or more transition alumina precursors, transition aluminas, or other alpha-alumina precursors, the porous body precursors and/or shaped porous bodies may desirably be fluoride affected, as may be achieved by exposing the porous body precursors and/or shaped porous bodies to fluorine-containing species, as may be provided in gaseous form, in gaseous or liquid solution, or via the provision of one or more solid fluorine-containing sources operatively disposed relative to the porous body precursors and/or shaped porous bodies. For advantages provided in processing, any such fluoride effect may desirably be achieved via exposure of the porous body precursors and/or shaped porous bodies to one or more fluorine-containing species in gaseous form or in gaseous solution. The particulars of such gaseous fluoride affectation are described in copending, commonly assigned PCT application no. PCT/US2006/016437, the entire disclosure of which is hereby incorporated by reference herein for any and all purposes.

One preferred method of providing the fluoride effect to the porous body precursors or shaped porous bodies comprises heating a vessel containing porous body precursors comprising the precursor alumina blend to a temperature of from about 750° C. to about 1150° C., preferably from about 850° C. to about 1050° C. A fluorine-containing gas is then introduced into the vessel and can establish a partial pressure within the vessel of between about 1 torr and about 10,000 torr. The partial pressure may be 1, 100, 200, 300, 400, 500, 600, 700, 800, 900, 1000, 2500, 5000, 7500, or 10,000 ton or pressures in between. Preferred partial pressures are below about 760 torr. The porous body precursors are allowed to be in contact with the fluorine-containing gas for a time of about 1 minute to about 48 hours. The time may be 1 minute, 15 minutes, 30 minutes, 45 minutes, 1 hour, 90 minutes, 2 hours, 3 hours, 4 hours, 5 hours, 10 hours, 20 hours, 30 hours, 40 hours or about 48 hours or any amount of time in between. Shorter times for contacting the gas with the porous body precursors are preferred, with times of from about 30 minutes to about 90 minutes being particularly preferred. Of course, and as those of ordinary skill in the art can readily appreciate, the preferred combinations of time and temperature and/or pressure vary with the fluorine-containing gas used, the precursor alumina blend added to the porous body precursors, and any other components of the porous body precursors.

One particularly preferred method of providing a fluoride effect to porous body precursors comprising one or more transition alumina precursors, transition aluminas or other alpha-alumina precursors, comprises heating a vessel containing the porous body precursors to a first temperature in the range of about 850° C. to about 1150° C. prior to introducing the fluorine-containing gas and then heating to a second temperature greater than the first temperature and between about 950° C. and about 1150° C. after introducing the fluorine-containing gas. Desirably, in these embodiments of the invention, the first temperature is increased to the second temperature at a rate of about 0.2° C. to about 4° C. per minute. Whatever time and temperature combination utilized, at least 50% of the transition alumina precursors, transition aluminas or other alpha-alumina precursors are desirably converted to alpha-alumina platelets.

Another particular method for preparing porous body precursors suitable for the preparation of shaped porous bodies desirably comprising fluoride-affected alpha-alumina comprises providing the germanium containing composition and/or selecting the precursor aluminas and mixing these to provide the germanium/precursor alumina blend, peptizing the germanium/precursor alumina blend with a mixture containing an acidic component and halide anions (preferably fluoride anions), forming (e.g., by extruding or pressing) the precursor alumina blend, and then drying and calcining the porous body precursors at temperatures between about 1000° C. and about 1400° C. for a time between about 45 minutes and about 5 hours to provide shaped porous bodies comprising fluoride-affected alpha-alumina.

Shaped porous bodies comprising alpha-alumina according to the invention will desirably have measured surface areas of at least about 0.5 m²/g (more preferably from about 0.7 m²/g to about 10 m²/g), measured pore volumes of at least about 0.5 cc/g (more preferably from about 0.5 cc/g to about 2.0 cc/g), purity of at least about 90 percent alpha-alumina particles, preferably at least about 95 percent alpha-alumina particles, and more preferably at least about 99 weight percent alpha-alumina particles, the shaped porous bodies also desirably having a median pore diameter from about 1 micron to about 50 microns. Further, the shaped porous bodies according to the invention will desirably be comprised largely of particles in the form of platelets, the term “platelet” being defined herein as a particle that has at least one, or two or more, substantially flat major surface(s). Desirably, at least 50 percent of the platelets (by number) will have a major dimension of less than about 50 microns. The “substantially flat major surface” referred to herein may be characterized by a radius of curvature of at least about twice the length of the major dimension of the surface.

Otherwise, the shaped porous bodies may comprise any suitable shape, as will depend upon the end use of the same. Like the porous body precursors, generally suitable shapes for the shaped porous bodies can include without limitation pills, chunks, tablets, pieces, spheres, pellets, tubes, wagon wheels, toroids having star shaped inner and outer surfaces, cylinders, hollow cylinders, amphora, rings, Raschig rings, honeycombs, monoliths, saddles, cross-partitioned hollow cylinders (e.g., having at least one partition extending between walls) cylinders having gas channels from side wall to side wall, cylinders having two or more gas channels, and ribbed or finned structures. If cylinders, the shaped porous bodies may be circular, oval, hexagonal, quadrilateral, or trilateral in cross-section. In those embodiments of the invention wherein the shaped porous bodies are used to prepare catalysts, the shaped porous bodies may desirably be formed into a rounded shape, e.g., pellets, rings, tablets and the like, having diameters of from about 0.1 inch (0.25 cm) to about 0.8 inch (2 cm).

In some embodiments, the shaped porous bodies may desirably be washed to remove any soluble residues thereon prior to the deposition of the components of the end-use product based thereupon. There is some indication that washed shaped porous bodies may exhibit at least marginally enhanced performance, although unwashed shaped porous bodies are also often successfully used in end-use products. If washing is desired, the shaped porous bodies may be washed with hot, e.g., from about 80° C. to about 100° C., demineralized water until the electrical conductivity of the effluent water does not decrease.

Once substantially fully processed, the shaped porous bodies may be subjected to a heat treatment step that can “fine tune” the physical, or morphological properties of the shaped porous bodies. That is, the beneficial effects of the germanium are not limited to those obtained during synthesis, and additional platelet growth and morphological changes may be observed if the fully processed shaped porous bodies are subjected to a heat treatment. More specifically, additional morphological changes may be seen by exposing the shaped porous bodies to an environment heated to a temperature of at least about 1000° C., or 1200° C., or even 1400° C., for time periods of from about 1 minute to about 12 hours, or from about 15 minutes to about 6 hours, or even from about 30 minutes to about 4 hours. Additional morphological changes may even be seen in periods as short of from about 1 hour to about 2 hours.

The heat treating atmosphere existing in the furnace can be 100% inert, i.e. nitrogen, argon or vacuum, the heat treating atmosphere can be 90% inert and 10% ambient air atmosphere, or the heat treating atmosphere can even be more oxidative comprising 100% ambient air atmosphere.

As mentioned, such heat treatment has now been surprisingly discovered to be capable of providing additional platelet growth or morphological changes to otherwise completely processed shaped porous bodies. That is, the heat treatment may provide improvements in one or more of surface area, aspect ratio, pore volume, median pore diameter, surface morphology, crush strength, yield or failure stress, calcined density, etc. In some embodiments, at least crush strength and median pore diameter may be improved by subjecting the shaped porous bodies to the heat treatment described herein, and in others, at least crush strength may be improved.

Because of their advantageous, enhanced mechanical properties, the shaped porous bodies provided by the invention are particularly well suited for incorporation into many end-use applications. More particularly, shaped porous bodies of the invention are well suited for use as, e.g., catalyst supports, filters, membrane reactors and preformed bodies for composites. As used herein, “carrier” and “support” are interchangeable terms. A carrier provides surface(s) to deposit, for example, catalytic metals, metal oxides, or promoters that are components of a catalyst.

Properties of a catalyst based upon the shaped porous bodies may also be enhanced via the inclusion in the porous body precursors of germanium and/or the precursor alumina blend, and these include selectivity, activity, lifetime, and the like. The “selectivity” of an epoxidation reaction, which is synonymous with “efficiency,” refers to the fraction, expressed as a percentage, of converted or reacted olefin that forms a particular product. The terms “efficiency” and “selectivity” are used interchangeably herein. The “activity” of an epoxidation reaction can be quantified in a number of ways, one being the mole percent of olefin oxide contained in an outlet stream of the reactor relative to that in an input stream (the mole percent of olefin oxide in the inlet stream typically, but not necessarily, approaches zero percent) while the reactor temperature is maintained substantially constant; and another being the temperature required to maintain a given rate of olefin oxide production. In many instances, activity is measured over a period of time in terms of the mole percent of olefin oxide produced at a specified constant temperature. Alternatively, activity can be measured as a function of the temperature required to sustain production of a specified constant mole percent of olefin oxide. One measure of the useful life, or “lifetime” of a catalyst, is the length of time that reactants can be passed through the reaction system during which time acceptable productivity is obtained in light of all relevant factors. “Deactivation”, as used herein, refers to a permanent loss of activity and/or efficiency, that is, a decrease in activity and/or efficiency that cannot be recovered. Generally, deactivation tends to proceed more rapidly when higher reactor temperatures are employed. The “stability” of a catalyst is inversely proportional to the rate of deactivation. Lower rates of deactivation are generally desirable.

If used as catalyst supports, the shaped porous bodies may advantageously be used as supports for catalysts useful for the epoxidation of alkenes, partial oxidation of methanol to formaldehyde, partial selective oxidation of saturated hydrocarbons to olefins, selective hydroformylation of olefins, selective hydrogenations, selective hydrogenation of acetylenes in cracked hydrocarbon streams, selective hydrogenation of di-olefins in olefin-di-olefin-aromatic streams also known as pyrolysis gasoline, and selective reduction of NOₓ to N₂. Other catalytic applications for the present shaped porous bodies include their use as carriers for automotive exhaust catalysts for emissions control and as carriers for enzymatic catalysis. In addition to end-use applications as catalytic supports, the inventive shaped porous bodies may also be used for the filtration of materials from liquid or gas streams, see, e.g. Auriol, et al., U.S. Pat. No. 4,724,028. In these applications the shaped porous bodies may either be the discriminating material, or may be the carrier for the discriminating material. Other uses for the present shaped porous bodies include, but are not limited to, packing for distillations and catalytic distillations.

Indeed, due to the numerous advantages imparted by the inventive shaped porous bodies to this particular end use, in one embodiment of the invention, the shaped porous body is used as the basis for a catalyst and these catalysts, as well as processes for making them, are also provided. Typically, such processes include at least selecting a shaped porous body prepared from a germanium-doped porous body precursor, which in some embodiments may further comprise a precursor alumina blend and depositing one or more catalytic species on the shaped porous body.

Once deposited, the catalytic species can be bound directly on the surface of the shaped porous bodies of the invention, or, the catalytic species may be bound to a washcoat, i.e., another surface which has been applied to the surface of the shaped porous bodies. The catalytic species may also be covalently attached to a macromolecular species, such as synthetic polymer or a biopolymer such as a protein or nucleic acid polymers, which in turn, is bound either directly to the surface of the shaped porous bodies or a washcoat applied thereto. Further, a deposited catalytic species may reside on the surface of the shaped porous bodies, be incorporated into a lattice provided on the surface of the shaped porous bodies, or be in the form of discrete particles otherwise interspersed among the shape porous bodies.

If the shaped porous bodies are desirably used as supports for catalysts, any catalytic species may be deposited thereupon. Non-limiting examples of catalytic species that may advantageously be supported by the shaped porous bodies include metals, solid state compounds, molecular catalysts, enzymes and combinations of these.

Metals capable of exhibiting catalytic activity include noble metals, e.g. gold, platinum, rhodium, palladium, ruthenium, rhenium, and silver; base metals such as copper, chromium, iron, cobalt, nickel, zinc, manganese, vanadium, titanium, scandium, and combinations of these. Solid state compounds suitable for use as catalytic species include, but are not limited to oxides, nitrides and carbides, and one particular example of a class of solid state compounds useful as a catalytic species are the perovskite-type catalysts that comprise a metal oxide composition, such as those described by Golden, U.S. Pat. No. 5,939,354, incorporated herein by reference. Exemplary molecular catalytic species include at least metal Schiff base complexes, metal phosphine complexes and diazaphosphacycles. Non-limiting examples of enzymes useful as catalytic species include lipases, lactases, dehalogenases or combinations of these, with preferred enzymes being lipases, lactases or combinations thereof. Typically, metals are utilized as the catalytic species in catalysts contemplated for use in epoxidation processes and silver in particular, is preferred.

The desired catalytic species may be deposited onto the shaped porous bodies according to any suitable method, to provide catalysts according to the invention. Molecular and enzymatic catalysts may typically be provided onto the shaped porous bodies via covalent attachment directly to the shaped porous bodies, to a wash coat (such as silica, alumina, or carbon) or supported high surface area carbon (such as carbon nanotubes) applied thereto. Enzyme catalysts may also be supported by other supports known in the art, including the carbon nanofibers such as those described by Kreutzer, WO2005/084805A1, incorporated herein by reference, polyethylenimine, alginate gels, sol-gel coatings, or combinations thereof. Molecular catalyst may also be immobilized on the surface(s) of the shaped porous bodies by any of the immobilization techniques generally known to those skilled in the art, such as attachment through silane coupling agents. Typically, metal catalytic species are conveniently applied by solution impregnation, physical vapor deposition, chemical vapor deposition or other techniques. Silver is typically deposited on shaped porous bodies to form epoxidation catalysts via solution impregnation and the same is contemplated here.

Typically, the shaped porous bodies will be impregnated with one or more silver compound solutions sufficient to allow the silver to be provided on the shaped porous bodies in an amount greater than about 5 percent, greater than about 10 percent, greater than about 15 percent, greater than about 20 percent, greater than about 25 percent, preferably, greater than about 27 percent, and more preferably, greater than about 30 percent by weight, based on the weight of the catalyst. Although the amount of silver utilized is not particularly limited, the amount of silver provided in connection with the shaped porous bodies may usually be less than about 70 percent, and more preferably, less than about 50 percent by weight, based on the weight of the catalysts.

In terms of density, the amount of catalytic species, e.g., silver, relative to the surface area of the shaped porous bodies may be about 0.10 g/m², or up to about 0.12 g/m², or up to about 0.15 g/m², or up to about 0.20 g/m², or up to about 0.40 g/m², or even up to about 0.50 g/m², or even 0.65 g/m².

Although silver particle size in the finished catalysts is important, the range is not narrow. A suitable silver particle size can be in the range of from about 10 angstroms to about 10,000 angstroms in diameter. A preferred silver particle size ranges from greater than about 100 angstroms to less than about 5,000 angstroms in diameter. It is desirable that the silver be relatively uniformly dispersed within, throughout, and/or on the shaped porous body.

Catalysts according to the present invention are based upon shaped porous bodies comprising germanium and/or a precursor alumina blend, and may also desirably comprise rhenium. The inventive catalysts may further include, in certain embodiments, one or more additional promoters, such as, e.g., cesium. Rhenium promoted, supported silver-containing catalysts are known from U.S. Pat. No. 4,761,394 and U.S. Pat. No. 4,766,105, which are incorporated herein by reference. Broadly, the catalysts comprise silver, rhenium or compound thereof, and in some embodiments, a co-promoter such as a further metal or compound thereof and optionally an additional co-promoter such as one or more of sulfur, phosphorus, boron, and compounds thereof, on the support material.

As is known to those skilled in the art, there are a variety of known promoters, or materials which, when present in combination with particular catalytic materials, e.g., silver, benefit one or more aspects of catalyst performance or otherwise act to promote the catalyst's ability to make a desired product, e.g., ethylene oxide or propylene oxide. More specifically, and while such promoters in themselves are generally not considered catalytic materials, they typically may contribute to one or more beneficial effects of the catalysts' performance, for example enhancing the rate, or amount, of production of the desired product, reducing the temperature required to achieve a suitable rate of reaction, reducing the rates or amounts of undesired reactions, etc. Furthermore, and as those of ordinary skill in the art are aware, a material which can act as a promoter of a desired reaction can be an inhibitor of another reaction. For purposes of the present invention, a promoter is a material which has an effect on the overall reaction that is favorable to the efficient production of the desired product, whether or not it may also inhibit any competing reactions that may simultaneously occur.

Known promoters for silver-based, epoxidation catalysts, in addition to rhenium, include, but are not limited to, molybdenum, tungsten, lithium, sulfur, sodium, manganese, rubidium, and cesium. Rhenium, molybdenum, sulfur or tungsten may suitably be provided as oxyanions, for example, as perrhenate, molybdate, sulfate or tungstate, in salt or acid form. Examples of promoters, their characteristics, and methods for incorporating the promoters as part of the catalyst are described in Thorsteinson et al., U.S. Pat. No. 5,187,140, particularly at columns 11 through 15, Liu, et al., U.S. Pat. No. 6,511,938, Chou et al., U.S. Pat. No. 5,504,053, Soo, et al., U.S. Pat. No. 5,102,848, Bhasin, et al., U.S. Pat. Nos. 4,916,243, 4,908,343, and 5,059,481, and Lauritzen, U.S. Pat. Nos. 4,761,394, 4,766,105, 4,808,738, 4,820,675, and 4,833,261, all incorporated herein by reference in their entirety for any and all purposes.

Catalysts comprising silver as a catalytic species as well as at least rhenium as a promoter are expected to find particular benefit when the present inventive shaped porous bodies, comprising germanium and/or a precursor alumina blend, are used as the bases thereof. The rhenium component can be provided in various forms, for example, as the metal, as a covalent compound, as a cation or as an anion. The rhenium species that provides the enhanced efficiency and/or activity is not certain and may be the component added or that generated either during preparation of the catalyst or during use as a catalyst. Examples of rhenium compounds include the rhenium salts such as rhenium halides, the rhenium oxyhalides, the rhenates, the perrhenates, the oxides and the acids of rhenium. However, the alkali metal perrhenates, ammonium perrhenate, alkaline earth metal perrhenates, silver perrhenates, other perrhenates and rhenium heptoxide may also be used. Rhenium heptoxide, Re₂O₇, when dissolved in water, hydrolyzes to perrhenic acid, HReO₄, or hydrogen perrhenate. Thus, for purposes of this specification, rhenium heptoxide can be considered to be a perrhenate, that is, ReO₄. Similar chemistries can be exhibited by other metals such as molybdenum and tungsten.

In some embodiments, catalysts comprising silver and rhenium may additionally comprise a promoting amount of at least one further metal and optionally a co-promoter. More specifically the further metal may be selected from the group of molybdenum, tungsten, chromium, titanium, hafnium, zirconium, vanadium, thallium, thorium, tantalum, niobium, gallium and mixtures thereof. Preferably the further metal is selected from the Group IA metals such as lithium, potassium, rubidium and cesium and/or from the Group IIA metals such as calcium and barium. More preferably it is lithium and/or cesium. Most preferably, it is cesium. Where possible, rhenium, the further metal or the co-promoter is provided as an oxyanion, in salt or acid form. Other optional co-promoters include, but are not limited to: tungsten, sodium, manganese, molybdenum, chromium, sulfur, phosphorous, boron, and mixtures thereof.

In some embodiments, catalyst can comprise at least a rhenium promoter, a first co-promoter, and a second co-promoter; where the quantity of the rhenium promoter deposited on the carrier is greater than 1 mmole/kg, relative to the weight of the catalyst; where the first co-promoter is selected from sodium, sulfur, phosphorus, boron, and mixtures thereof; where the second co-promoter is selected from tungsten, molybdenum, chromium, manganese and mixtures thereof; and the total quantity of the first co-promoter and the second co-promoter deposited on the carrier can be at least about 3.5 mmole/kg, or at least about 4.5 mm/kg, or even up to about 6.0 mmole/kg, or even greater, relative to the weight of the catalyst.

The rhenium and any other desired promoters included in the catalyst are desirably provided in a promoting amount, and such amounts are readily determined by those of ordinary skill in the art. The concentration of the one or more promoters present in the catalyst may vary over a wide range depending on the desired effect on catalyst performance, the other components of a particular catalyst, the physical and chemical characteristics of the carrier, and the epoxidation reaction conditions.

A “promoting amount” of a certain promoter refers to an amount of that promoter that works effectively to provide an improvement in one or more of the properties of a catalyst comprising the promoter relative to a catalyst not comprising said promoter. Examples of catalytic properties include, inter alia, selectivity, activity, lifetime, stability, etc.

The promoting effect provided by the promoters can be affected by a number of variables such as for example, reaction conditions, catalyst preparative techniques, surface area and pore structure and surface chemical properties of the support, the silver and co-promoter content of the catalyst, the presence of other cations and anions present on the catalyst. The presence of other activators, stabilizers, promoters, enhancers or other catalyst improvers can also affect the promoting effects. Generally speaking, promoting amounts of rhenium may be at least about 1 ppmw, at least about 5 ppmw, or between from about 10 ppmw to about 2000 ppmw, often between about 20 ppmw and 1000 ppmw, calculated as the weight of rhenium based on the total weight of the catalyst.

Other promoters and/or co-promoters vary in concentration from about 0.0005 to 1.0 wt. %, preferably from about 0.005 to 0.5 wt. %. For some, e.g., cationic promoters, amounts between about 10 ppm and about 4000 ppm, preferably about 15 ppm and about 3000 ppm, and more preferably between about 20 ppm and about 2500 ppm by weight of cation calculated on the total support material are appropriate. Amounts between about 50 ppm and about 2000 ppm are frequently most preferable. If cesium is used in mixture with other cations, the ratio of cesium to any other cation(s), may vary from about 0.0001:1 to 10,000:1, preferably from about 0.001:1 to 1,000:1.

Methods of preparing epoxidation catalysts are well-known in the art, and any of these are suitable for use in preparing the catalysts based upon the porous body precursors and shaped porous bodies. Generally speaking, the methods involve one or more impregnation steps with one or more solutions comprising the desired catalyst components. Typically, a reduction step is conducted during or after the impregnations, to form metallic silver particles. Thorsteinson et al., U.S. Pat. No. 5,187,140 describes methods of forming catalysts, and is incorporated herein by reference for any and all purposes.

One particular example of an epoxidation of commercial importance is the epoxidation of alkylenes, or mixtures of alkylenes. Many references describe these reactions, representative examples of these being Liu et al., U.S. Pat. No. 6,511,938 and Bhasin, U.S. Pat. No. 5,057,481, as well as the Kirk-Othmer's Encyclopedia of Chemical Technology, 4^{th} Ed. (1994) Volume 9, pages 915-959, all of which are incorporated by reference herein in their entirety for any and all purposes. Although the invention is not so limited, for purposes of simplicity and illustration, catalysts according to the invention useful in epoxidations will be further described in terms of, and with reference to, the epoxidation of ethylene.

Catalysts are a very important factor in the commercial viability of such epoxidation reactions. The performance of catalysts in these reactions is typically evaluated on the basis of the catalysts' selectivity, activity, and stability during the epoxidation reactions. Stability typically refers to how the selectivity or activity of the process changes during the time that a particular batch of catalyst is being used, i.e., as more olefin oxide is produced. Catalysts of the present invention, based upon the porous body precursors and shaped porous bodies disclosed herein are expected to provide advantages in selectivity and/or activity resulting from one or more property changes provided by the porous body precursors and/or shaped porous bodies comprising an amount of germanium, as well as a precursor alumina blend.

Generally speaking then, the epoxidation reaction may take place in any suitable reactor, for example, fixed bed reactors, continuous stirred tank reactors (CSTR), and fluid bed reactors, a wide variety of which are well known to those skilled in the art and need not be described in detail herein. The desirability of recycling unreacted feed, employing a single-pass system, or using successive reactions to increase ethylene conversion by employing reactors in series arrangement can also be readily determined by those skilled in the art. The particular mode of operation selected is usually dictated by process economics. Conversion of olefin (alkylene), preferably ethylene, to olefin oxide, preferably ethylene oxide, can be carried out, for example, by continuously introducing a feed stream containing alkylene (e.g., ethylene) and oxygen or an oxygen-containing gas to a catalyst-containing reactor at a temperature of from about 200° C. to about 300° C., and a pressure which may vary between about 5 atmospheres (506 kPa) and about 30 atmospheres (3.0 MPa), depending upon the mass velocity and productivity desired. Residence times in large-scale reactors are generally on the order of from about 0.1 seconds to about 5 seconds. Oxygen may be supplied to the reaction in an oxygen-containing stream, such as, air or as commercial oxygen, or as oxygen-enriched air. The resulting alkylene oxide, preferably, ethylene oxide, is separated and recovered from the reaction products using conventional methods.

Any alkylene can be utilized in the process, and examples of those that may desirably be epoxidized include, but are not limited to, 1,9-decadiene, 1,3-butadiene, 2-butene, isobutene, 1-butene, propylene, ethylene, or combinations of these. Preferably, the alkylene comprises ethylene.

Typically, epoxidation reactions may desirably be carried out in the gas phase, with a feed comprising the desired alkylene and oxygen being caused to come in contact with an epoxidation catalyst. Oftentimes, the catalyst is present as a solid material, and more particularly, may be present as a packed bed within the desired reactor. The quantity of catalyst used may be any suitable amount and will depend upon the application. In pilot plant reactors, the quantity of catalyst may be, e.g., less than about 5 kg, while in commercial epoxidation plants, the quantity of catalyst used in the packed bed may be at least about 10 kg, or at least 20 kg, or from about 10² to 10⁷ kg or from about 10³ to 10⁶ kg.

Many epoxidation reactions are carried out as continuous processes, and the same is contemplated here. In such processes, the desired reactor may typically be equipped with heat exchange equipment to control the temperature of the process, within the reactor and/or the catalyst bed.

In one embodiment, the process for the oxidation of an alkylene comprises contacting a reaction mixture feed comprising an alkene, oxygen, and carbon dioxide, with a catalyst comprising a carrier and, deposited on the carrier, silver, a rhenium promoter, a first co-promoter, and a second co-promoter; wherein the carbon dioxide is present in the reactor mixture in a quantity of at most 3 mole percent based on the total reaction mixture; the first co-promoter is selected from sulfur, phosphorus, boron, and mixtures thereof; and the second co-promoter is selected from tungsten, molybdenum, chromium, and mixtures thereof.

The alkylene oxide produced by the present epoxidation process may typically be processed to provide further downstream products, such as, for example, 1,2-diols, 1,2-diol ethers, 1,2-carbonates, and alkanolamines. Since the present invention provides an improved epoxidation method, it is contemplated that the improvements provided will carry forward to provide improvements to these downstream processes and/or products. Improved methods for the production of 1,2-diols, 1,2-diol ethers, 1,2-carbonates, and alkanolamines are thus also provided herein.

The conversion of alkylene oxides into 1,2-diols or 1,2-diol ethers may comprise, for example, reacting the desired alkylene oxide with water, suitably in the presence of an acidic or basic catalyst. For example, for preferential production of the 1,2-diol over the 1,2-diol ether, the alkylene oxide may be reacted with a tenfold molar excess of water, in a liquid phase reaction in the presence of an acid catalyst, e.g., 0.5-1.0 wt % sulfuric acid, based on the total reaction mixture, at 50° C. to about 70° C. at 1 bar absolute, or in a gas phase reaction, at 130° C. to about 240° C. and from about 20 bar to about 40 bar absolute, preferably in the absence of a catalyst. If the proportion of water is lowered, the proportion of the 1,2-diol ethers in the reaction mixture will be increased. The 1-2, diol ethers thus produced may comprise di-ethers, tri-ethers, tetra-ethers or other multi-ethers. Alternative 1,2-diol ethers may be prepared by converting the alkylene oxide with an alcohol, such as methanol or ethanol, or by replacing at least a portion of the water with the alcohol. The resulting 1,2-diols and diol ethers may be utilized in a wide variety of end-use applications in the food, beverage, tobacco, cosmetic, thermoplastic polymer, curable resin system, detergent, heat transfer system, etc., industries.

The conversion of alkylene oxides produced via the method of the present invention into alkanolamines may comprise, for example, reacting the alkylene oxide with ammonia. Anhydrous or aqueous ammonia may be used, although anhydrous ammonia favors the production of monoalkanolamine, and may be used when the same is preferred. The resulting alkanolamines may be used, for example, in the treatment of natural gas. The olefin oxide may be converted into the corresponding 1,2-carbonate by reacting the olefin oxide with carbon dioxide. If desired, a 1,2-diol may be prepared by subsequently reacting the 1,2-carbonate with water or an alcohol to form the 1,2-diol. For applicable methods, reference is made to U.S. Pat. No. 6,080,897, which is incorporated herein by reference.

The following examples further illustrate the invention, without limiting the scope thereof.

### Example 1

Preparation of Porous Precursor Bodies Comprising Germanium and a Precursor Alumina Blend

Sample #1-0.25% loading of germanium oxide:

500 grams of Catapal B alumina and 500 grams of Versal V-250 alumina were weighed and placed into a plastic bucket. 65 grams of A4M Methocel and 2.5 grams of germanium oxide were added to the alumina mixture. The dry ingredients were put into a Mix Muller and were blended for five minutes. 30 grams of oleic acid and 640 grams of deionized water were then slowly added to the dry ingredients and mulled for an additional ten minutes.

Sample #2-1.0% loading of germanium oxide:

500 grams of Catapal B alumina and 500 grams of Versal V-250 alumina were weighed and placed into a plastic bucket. 65 grams of A4M Methocel and 10 grams of germanium oxide were added to the alumina mixture. The dry ingredients were put into a Mix Muller and were blended for five minutes. 30 grams of oleic acid and 640 grams of deionized water were then slowly added to the dry ingredients and mulled for an additional ten minutes.

Sample #3-2.0% loading of germanium oxide:

500 grams of Catapal B alumina and 500 grams of Versal V-250 alumina were weighed and placed into a plastic bucket. 65 grams of A4M Methocel and 20 grams of germanium oxide were added to the alumina mixture. The dry ingredients were put into a Mix Muller and were blended for five minutes. 30 grams of oleic acid and 640 grams of deionized water were then slowly added to the dry ingredients and mulled for an additional ten minutes.

In each case, the resultant paste was extruded into 5/16 inch rings via a twin screw extruder.

The porous body precursors were then spread out in plastic pans and put in a 60° C. vented oven to dry for 4 days. After drying, the porous body precursors were calcined to 700° C. overnight.

Preparation of Shaped Porous Bodies from Porous Body Precursor Sample #'s 1-3

The Ge-doped porous body precursors were then separately converted to alpha alumina by the following process:

200 grams of the porous body precursors were placed into an alumina tube furnace reactor, a vacuum was pulled, and the reactor was heated to 840° C. overnight. The vacuum was turned off, and the reactor vessel was filled with HFC-134a (1,1,1,2-tetrafluoroethane) to a pressure of 300 ton. The furnace was held at 840° C. for 3 hours, then ramped at 2° C./min to 960° C. and held for an additional 2 hours. The gas was then removed from the reactor, the tube furnace was purged 3 times with nitrogen and cooled at a rate of 2° C./min to room temperature. The resultant shaped porous bodies, comprising alpha alumina platelets, were then removed from the reactor tube for analysis and catalyst preparation and testing.

Heat Treatment of Shaped Porous Bodies Sample #'s 1-3

A portion of each sample of shaped porous bodies was exposed to a heated oxidative atmosphere as follows. The GeO₂-doped shaped porous bodies were placed into a non-sealed laboratory furnace under atmospheric conditions. The furnace was heated at 2° C./min to 1400° C., held for 4 hours, and then cooled at 2° C./min back to room temperature.

Characterization of Shaped Porous Bodies Sample #'s 1-3

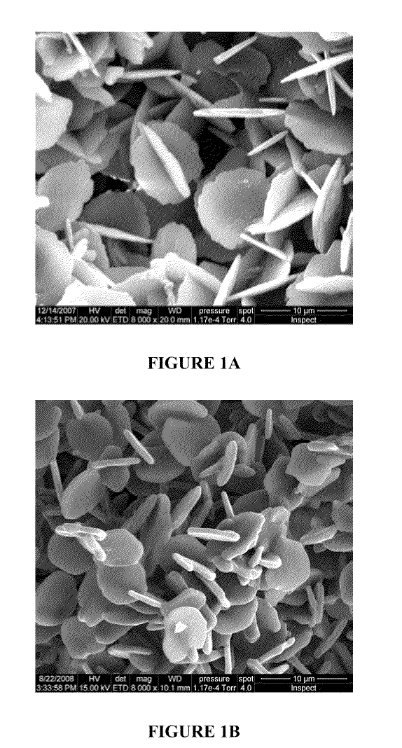

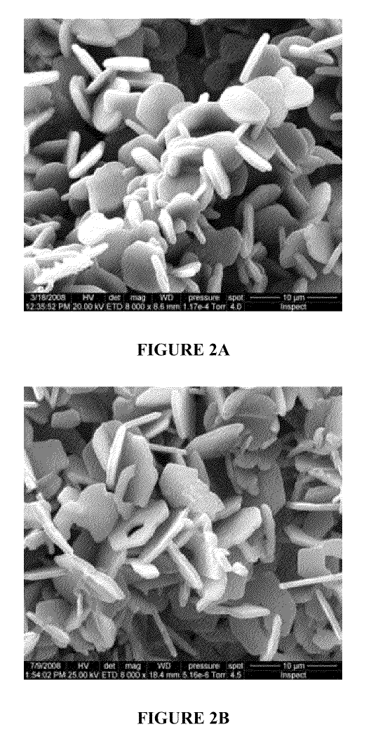

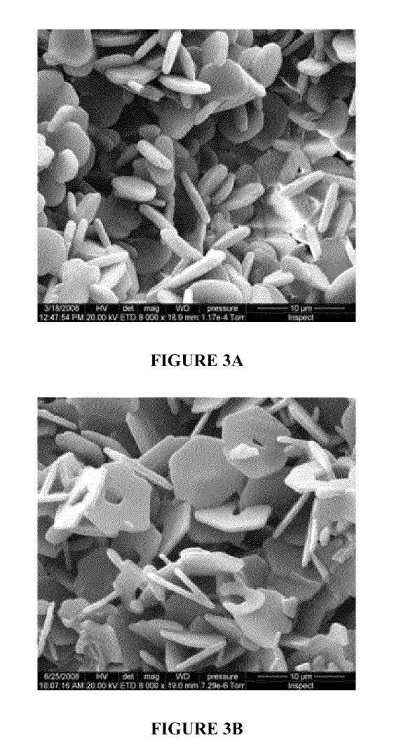

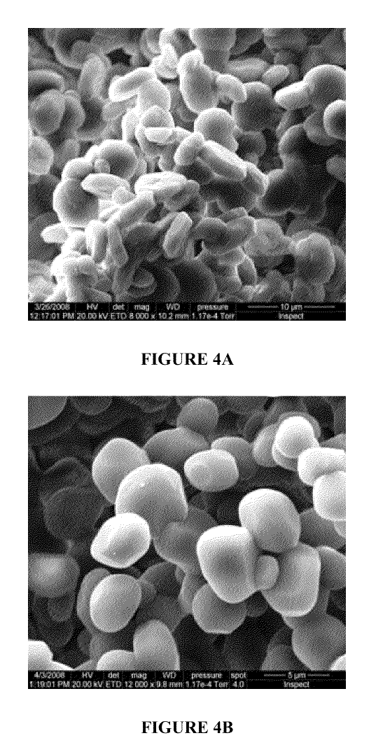

Shaped porous bodies were characterized as synthesized, as well as after heat treatment at 1400° C. and 1500° C., via a Quanta Inspect SEM. SEM images of as-synthesized and 1400° C. heat treated comparative shaped porous bodies, i.e., with no GeO₂ dopant, but comprising the same precursor alumina blend, are presented in FIGS. 1A and 1B. The images of the as-synthesized and heat treated inventive shaped porous bodies are presented in FIGS. 2A-4B. As shown, although platelet growth occurred during the heat treatment step for the samples containing lower levels of GeO₂ and gross morphology changes occurred with the heat treatment step for the 2% GeO₂ doped material (Samples 1-3), no visible enlargement of the platelet size due to the heat treatment was observed in the shaped porous bodies comprising only the precursor alumina blend.

Flat plate crush strength measurements were made with a Shimpo FGE-100X force gauge attached to the moving stage of a Mecmesin M1000 EC electronic screw drive system using the ASTM Standard D 6175-98 methodology. Values presented in Table 1 are averages for ten separate measurements. The 1400° C. heat treatment of the 0.25% and 1% GeO₂ doped carriers resulted in 50% and 54% increases in average crush strength whereas the same heat treatment of the comparative shaped porous bodies containing no GeO₂ dopant resulted in only a 31% increase in average crush strength.

TABLE 1

| | Heat | | |
|---|---|---|---|
| | Treatment, | Average Crush | Standard Deviation, |
| Sample ID | ° C. | Strength, lbs/mm | lbs/mm |
| 1 | None | 1.47 | 0.54 |
| 1 | 1400° C. | 2.34 | 0.38 |
| 2 | None | 1.27 | 0.45 |
| 2 | 1400° C. | 1.96 | 0.59 |
| 3 | None | 0.11 | 0.07 |
| 3 | 1500° C. | 0.42 | 0.08 |
| Comparative* | None | 1.92 | 0.20 |
| Comparative* | 1400° C. | 2.51 | 0.33 |

*Comprises same precursor alumina blend, but no germanium.

Mercury porosimetry characterization of the shaped porous bodies was completed with a Micromeritics Autopore IV 9520 after outgassing under vacuum at ambient temperature. These results are summarized in Table 2. Addition of increasing levels of the GeO₂ promoter gave final shaped porous bodies with larger median pore diameters than the comparative shaped porous bodies.

TABLE 2

| | Total Pore | Median Pore |
|---|---|---|
| Sample ID | Volume, mL/g | Dia. (Vol.), μm |
| 1 | 0.6717 | 2.8095 |
| 1, heat treated | 0.6629 | 2.6282 |
| 2 | 0.6713 | 3.4961 |
| 2, heat treated | 0.6725 | 3.7102 |
| 3, heat treated | 0.6342 | 5.2652 |
| Comparative, heat | 0.6892 | 2.9676 |
| treated | | |

XPS results for the shaped porous body samples are summarized in Table 3. Detectable levels of germanium were not measured on the surface of the shaped porous body samples as measured by XPS. However, neutron activation did confirm trace amounts of germanium in the doped shaped porous bodies, thereby illustrating that the germanium oxide can exert its beneficial effects at low levels. XPS measurements were not completed on the 2% GeO₂ sample. XPS measurements for the other germanium doped shaped porous bodies were performed on a Physical Electronics Model 5600 Multi-technique Surface Analysis System. The analyses were performed using a monochromator aluminum X-ray source (Kα=1486.6 eV) at 400 watts (15 KeV and 26.7 mA current). The signal was acquired on an 800 μm diameter area for general surface characterization. The XPS survey and high-resolution spectra were acquired at 187.5 eV and 11.75 eV pass energies, respectively. The C1s peak at 284.8 eV was used as binding energy (Eb) charge reference. XPS characterization of the undoped comparative shaped porous bodies were completed with a Kratos Axis HSi X-ray Photoelectron Spectrometer instrument using monochromatic Al Ka source operating at 14 kV and 15 mA.

TABLE 3

| Sample ID | O | C | Al | Si | F | Na | Ca | Mg | Ti |
|---|---|---|---|---|---|---|---|---|---|
| 1 | 48.0 | 10.5 | 30.2 | 0.3 | 9.4 | 1.1 | 0.2 | 0.3 | |
| 1HT* | 57.9 | 7.8 | 29.7 | 0.8 | 0.0 | 1.3 | 0.8 | 0.0 | |
| 2 | 48.1 | 13.2 | 28.6 | 0.5 | 6.9 | 1.7 | 0.3 | 0.3 | |
| 2HT* | 55.9 | 10.3 | 28.7 | 1.1 | 0.0 | 1.3 | 0.8 | 0.0 | |
| Comparative | 49.2 | 5.0 | 33.8 | | 11.6 | nd | 0.3 | | nd |
| ComparativeHT* | 55.0 | 7.7 | 32.5 | | 2.8 | 0.1 | 0.6 | | 1.3 |

*HT = Heat treated

Preparation of Catalysts based upon Shaped Porous Body Sample #'s 1-3

The shaped porous bodies were vacuum impregnated with a silver solution containing approximately 28 wt % silver oxide, 18 wt % oxalic acid dihydrate, 17 wt % ethylenediamine, 6 wt % monoethanolamine, and 31% water. The shaped porous bodies were loaded into a vacuum vessel and evacuated for 15-30 minutes, maintaining a minimum of 28 inches of Hg. After the evacuation, the impregnation solution was slowly added to carrier by opening the stopcock of a reparatory funnel located at the top of the vacuum impregnation column. The vacuum was released after the impregnation solution had been added. The shaped porous bodies were submerged in the impregnation solution for 15 minutes with the excess impregnation solution drained from the vessel after the shaped porous body exposure was completed.

The impregnated shaped porous bodies/catalysts were spread into a single layer on a stainless steel mesh tray and placed on the mesh belt of the catalyst roaster. The catalysts were exposed to a hot zone of 500° C. with 266 scfh air flow through the bed for a period of about 2.5 minutes. After completion, the catalysts were cooled to room temperature and weighed to determine the Ag loading after the first impregnation step.

Additional promoter salts were added to the Ag impregnation solution including cesium hydroxide, lithium acetate, sodium acetate, ammonium perrhenate, ammonium sulfate, manganous nitrate, and diammonium EDTA prior to its use for the second impregnation. The same impregnation and roasting methodology were used for the second impregnation step of the inventive catalysts. The comparative catalyst was prepared using similar techniques.

The weight gains from both impregnation steps were used to calculate the catalyst formulations from the catalyst recipes based upon the inventive shaped porous bodies and porous body precursors. The values listed in Table 4 for the comparative catalyst were measured by XRF and the concentration of lithium and sodium were not determined using this technique.

TABLE 4

| Sample | Catalyst | Wt % | ppm | ppm | ppm | ppm | ppm | ppm |
|---|---|---|---|---|---|---|---|---|
| ID | ID | Ag | Cs | Li | Na | Re | SO₄ | Mn |
| 1 | 1A | 36.28 | 446 | 23 | 29 | 313 | 112 | 92 |
| 1 | 1B | 36.16 | 522 | 27 | 33 | 366 | 130 | 107 |
| 1 | 1C | 35.79 | 684 | 35 | 44 | 480 | 171 | 141 |
| 1HT* | 1D | 36.57 | 457 | 24 | 29 | 320 | 114 | 94 |
| 1HT* | 1E | 35.84 | 509 | 26 | 32 | 356 | 127 | 105 |
| 1HT* | 1F | 35.80 | 685 | 35 | 44 | 480 | 171 | 140 |
| 2HT* | 2A | 35.83 | 430 | 22 | 28 | 301 | 107 | 89 |
| 2HT* | 2B | 35.67 | 501 | 26 | 32 | 350 | 125 | 103 |
| 2HT* | 2C | 35.68 | 677 | 35 | 43 | 474 | 169 | 140 |
| 3HT\hat{ } | 3A | 36.00 | 207 | 11 | 14 | 145 | 51 | 43 |
| 3HT\hat{ } | 3B | 35.54 | 237 | 12 | 15 | 165 | 59 | 49 |
| 3HT\hat{ } | 3C | 35.87 | 327 | 17 | 21 | 228 | 81 | 68 |
| CompHT* | 4A | 36.62 | 572 | 30 | 37 | 401 | 142 | 118 |

HT* = Heat treatment, 1400° C.
HT\hat{ } = Heat treatment, 1500° C.

Testing of Catalysts

Catalyst samples were crushed with a motorized motar and pestle and sized using standard sieves to a 30/50 mesh cut.

130 μL each catalyst was loaded into a reactor well of a 48 channel Sinteff high throughput reactor. The catalysts were tested at 300 psig at 230° C. with the following feed composition: 10% CH₄, 35% C₂H₄, 0.6% C₂H₆, 2.5% CO₂, 7% O₂ He balance at 9000 hr-1 space velocity. ethyl chloride was added at different levels in a traverse to determine performance sensitivity after the catalysts had been on stream under constant conditions for 48 hours. The total time onstream for catalyst screening was 4.6 days. Catalyst performance was monitored using on-line Maxum GCs. Detailed results from the screening tests are summarized in Table 5. A high level overview of the results is also summarized graphically in FIG. 5. In general, the catalysts prepared on 0.25 and 1 wt % GeO₂ doped shaped porous bodies showed higher EO activity than that prepared on the corresponding undoped comparative shaped porous body, with equivalent to lower carbon efficiency.

TABLE 5

| Sample | Catalyst | EO, | EO |
|---|---|---|---|
| ID | ID | vol % | Selectivity, % |
| 1 | 1A | 2.3 | 82.3 |
| 1 | 1B | 1.9 | 85.0 |
| 1 | 1C | 1.6 | 87.6 |
| 1HT* | 1D | 2.0 | 83.3 |
| 1HT* | 1E | 1.9 | 82.1 |
| 1HT* | 1F | 2.0 | 84.8 |
| 2HT* | 2A | 1.9 | 83.1 |
| 2HT* | 2B | | |
| 2HT* | 2C | 1.7 | 84.6 |
| 3HT\hat{ } | 3A | 1.2 | 83.1 |
| 3HT\hat{ } | 3B | 1.1 | 83.0 |
| 3HT\hat{ } | 3C | 0.5 | 88.9 |
| CompHT* | 4A | 1.5 | 84.9 |

While only certain features of the invention have been illustrated and described herein, many modifications and changes will occur to those skilled in the art. The examples above further illustrate the invention, without limiting the scope thereof. It is to be understood that the appended claims are intended to cover all such modifications and changes as fall within the true spirit of the invention.

1. A germanium-doped porous body precursor comprising a precursor alumina blend.

2. The porous body precursor of claim 1, wherein the precursor alumina blend comprises a blend of at least two precursor aluminas.

3. The porous body precursor of claim 2, wherein the at least two precursor aluminas comprise gibbsite, bayerite, and nordstrandite, boehmite, pseudo-boehmite, diaspore, gamma-alumina, delta-alumina, eta-alumina, kappa-alumina, chi-alumina, rho-alumina, theta-alumina, aluminum trihydroxides and aluminum oxide hydroxides.

4. The porous body precursor of claim 3, wherein the at least two precursor aluminas comprise pseudo-boehmite or gibbsite.

5. The porous body precursor of claim 1, wherein the precursor alumina blend comprises a blend of two variants of one precursor alumina.

6. The porous body precursor of claim 1, wherein the precursor alumina blend comprises a blend of two secondary particles sizes of one precursor alumina.

7. The porous body of claim 1, further comprising methyl cellulose.

8. A shaped porous body prepared from a germanium-doped porous body precursor comprising a precursor alumina blend.

9. The shaped porous body of claim 8, wherein the shaped porous body comprises alpha-alumina.

10. The shaped porous body of claim 9, wherein the alpha-alumina is fluoride-affected.

11. A process for making a shaped porous body comprising preparing a germanium-doped porous body precursor, processing the porous body precursor into the shaped porous body, and exposing the shaped porous body to a heated inert or oxidative atmosphere.

12. The process of claim 11, wherein the inert or oxidative atmosphere is heated to a temperature of at least about 1000° C.

13. The process of claim 11, wherein the processing comprises adding methyl cellulose to the germanium-doped porous body precursor.

14. A rhenium-promoted catalyst comprising at least one catalytic species deposited on a shaped porous body, wherein the shaped porous body is prepared from a germanium-doped porous body precursor.

15. The rhenium-promoted catalyst of claim 14, wherein the catalytic species comprises a silver component.

16. A process for making a rhenium-promoted catalyst comprising:
a) selecting a shaped porous body prepared from a germanium-doped porous body precursor;
b) depositing at least one catalytic species and at least one promoter comprising rhenium on the shaped porous body.

17. A process for the epoxidation of an alkylene, comprising reacting a feed comprising one or more alkylenes and oxygen in the presence of a catalyst according to claim 14.

18. A process for preparing a 1,2-diol, a 1,2-diol ether, a 1,2-carbonate, or an alkanolamine comprising converting an alkylene oxide prepared by the process of claim 17 into the 1,2-diol, 1,2-diol ether, a 1,2-carbonate, or alkanolamine.

19. A catalyst comprising at least one catalytic species deposited on a shaped porous body, wherein the shaped porous body is prepared from a germanium-doped porous body precursor.

20. A process for making a catalyst comprising:
a) selecting a shaped porous body prepared from a germanium-doped porous body precursor;
b) depositing at least one catalytic species on the shaped porous body.
